(19) United States
(12) Patent Application Publication
(10) Pub. No.: US 2024/0096506 A1
(43) Pub. Date: Mar. 21, 2024

(54) NON-TRANSITORY COMPUTER READABLE MEDIUM

(71) Applicant: FUJIFILM Corporation, Tokyo (JP)

(72) Inventors: Keiji TSUBOTA, Kanagawa (JP); Noriaki IDA, Tokyo (JP); Shozo IYAMA, Kanagawa (JP)

(73) Assignee: FUJIFILM Corporation, Tokyo (JP)

(21) Appl. No.: 18/468,750

(22) Filed: Sep. 18, 2023

(30) Foreign Application Priority Data
Sep. 20, 2022 (JP) 2022-148937

(51) Int. Cl. G16H 80/00; A61B 5/00; A61B 5/11; G16H 40/67

(52) U.S. Cl. CPC G16H 80/00; A61B 5/1113; A61B 5/681; A61B 5/7282; G16H 40/67

(57) ABSTRACT

A health checkup support program according to an aspect of the present invention causes a central control unit of a smart watch that acquires biological information of a wearer to realize a diagnostic item detection function of detecting a diagnostic item of the wearer by accessing a health checkup information server that stores health checkup information related to the health checkup, and a diagnosis possibility determination function of determining whether or not at least one of the detected diagnostic items is appropriately implementable based on the biological information of the wearer obtained from the smart watch.

### CROSS-REFERENCE TO RELATED APPLICATIONS

This application claims priority under 35 U.S.C § 119(a) to Japanese Patent Application No. 2022-148937 filed on 20 Sep. 2022. The above application is hereby expressly incorporated by reference, in its entirety, into the present application.

### BACKGROUND OF THE INVENTION

### 1. Field of the Invention

The present invention relates to a non-transitory computer readable medium for storing a computer-executable program for causing a computer of a wearable device that acquires biological information of a wearer to realize a predetermined function.

### 2. Description of the Related Art

As the wearable device that acquires the biological information of the wearer, for example, a smart watch is known. Since the smart watch can acquire a body temperature, a heart rate, a blood pressure, a blood glucose level, and the like as the biological information of the wearer, it is effective for daily health management (see JP2022-003554A (corresponding to US2018/329584A1) and JP2022-037048A (corresponding to US2019/103007A1) below).

### SUMMARY OF THE INVENTION

As in JP2022-003554A and JP2022-037048A, measuring (collecting) the biological information with the wearable device is effective for daily health management, but in order to lead a healthier life, it is preferable to have a regular checkup by a doctor or the like, such as a health checkup.

However, there is a problem in that an accurate result cannot be obtained in a case in which the health checkup is performed in a state of a poor physical condition, such as a high fever state or a tense state (a state of high heart rate). Such a problem is expected to become more pronounced in countries that are less familiar with the health checkup, that is, countries other than Japan, where employers are not obliged to have their employees undergo the health checkup.

The present invention has been made in view of the above background, and an object of the present invention is to provide a non-transitory computer readable medium for storing a computer-executable program for enabling an appropriate health checkup not only in Japan, which is familiar with the health checkup, but also in other countries, which are less familiar with the health checkup.

In order to solve the problem, according to the present invention, there is provided a non-transitory computer readable medium for storing a computer-executable program for causing a computer of a wearable device that acquires biological information of a wearer to realize a predetermined function comprising: a diagnostic item detection function of detecting a diagnostic item on the day of a health checkup of the wearer by accessing a health checkup information storage unit that stores health checkup information related to the health checkup; and a diagnosis possibility determination function of determining whether or not at least one of the diagnostic items detected by the diagnostic item detection function is appropriately implementable by using the biological information of the wearer obtained via the wearable device.

It is preferable that the predetermined function includes a guidance function of providing guidance regarding the implementation of the diagnostic item based on a determination result of the diagnosis possibility determination function.

It is preferable that the guidance includes guidance on directions to an implementation place for a diagnostic item determined as being appropriately implementable.

It is preferable that the health checkup information storage unit stores location information of the implementation place, the wearable device acquires position information of the wearer, and the guidance function is a function of providing the guidance using the location information of the implementation place and the position information of the wearer.

It is preferable that the guidance includes guidance on an action for enabling implementation of a diagnostic item determined as not being appropriately implementable.

It is preferable that the predetermined function includes a notification function of, in a case in which it is determined that the diagnostic item is not appropriately implementable by the diagnosis possibility determination function, notifying a predetermined notification destination of the fact.

It is preferable that the predetermined notification destination is a terminal of a staff of an implementation facility of the health checkup.

It is preferable that, in a case in which a plurality of the diagnostic items are implemented in one health checkup, the determination is performed for each of the plurality of diagnostic items by the diagnosis possibility determination function, and the predetermined function includes an implementation order decision function of deciding an implementation order of the plurality of diagnostic items by using a determination result of each of the plurality of diagnostic items.

It is preferable that the determination by the diagnosis possibility determination function is repeatedly performed at a predetermined moment, and the predetermined function includes an implementation order change function of, in a case in which a diagnostic item determined as not being appropriately implementable in the determination occurs after the implementation order is decided, changing the implementation order.

It is preferable that, in addition to the biological information of the wearer, a current operating status and a future operating schedule of a medical resource required for the implementation of each of the diagnostic items are used to decide the implementation order.

It is preferable that the determination by the diagnosis possibility determination function is performed based on at least any of the past biological information of the wearer, an age, a sex, or a previous illness of the wearer.

According to the present invention, it is possible to perform an appropriate health checkup.

### BRIEF DESCRIPTION OF THE DRAWINGS

FIG. 1 is a schematic diagram showing a configuration of a health checkup support system.

FIG. 2 is an explanatory diagram showing contents of diagnostic item data.

FIG. 3 is an explanatory diagram showing contents of determination criterion data.

FIG. 4 is an explanatory diagram showing contents of health checkup progress information.

FIG. 5 is an explanatory diagram showing contents of implementation place data.

FIG. 6 is a block diagram showing a schematic configuration of a smart watch.

FIG. 7 is a flowchart showing a flow of determination as to whether or not implementation of a diagnostic item is appropriate.

FIG. 8 is a schematic diagram showing a configuration of a health checkup support system.

FIG. 9 is an explanatory diagram showing contents of medical resource operation information.

FIG. 10 is a block diagram showing a schematic configuration of a smart watch.

FIG. 11 is a flowchart showing a flow of decision and change of an implementation order.

### DESCRIPTION OF THE PREFERRED EMBODIMENTS

### First Embodiment

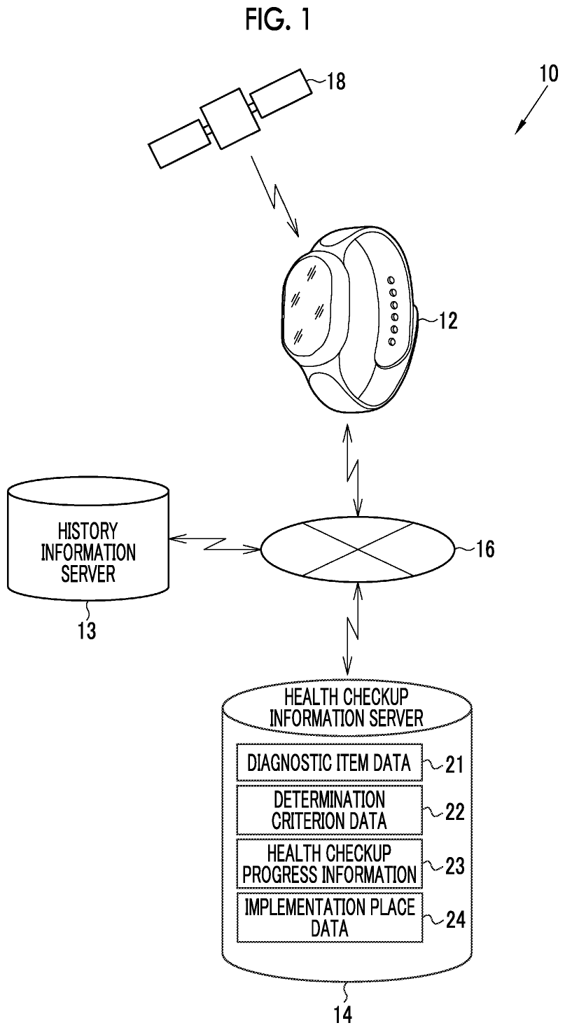

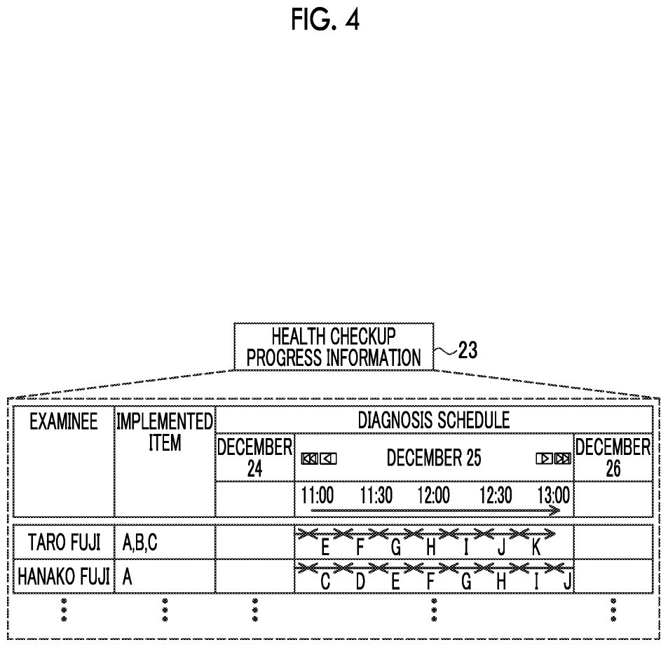

In FIG. 1, a health checkup support system 10 is for supporting an appropriate execution of a health checkup. The health checkup support system 10 comprises a smart watch 12 (wearable device) in which a health checkup support program 36 (see FIG. 4) according to an embodiment of the present invention is installed, a history information server 13, and a health checkup information server 14 (health checkup information storage unit), and these are communicably connected to each other via a network 16 such as the Internet.

The smart watch 12 is a watch-type wearable device to be worn on a wrist. The smart watch 12 may be owned by a wearer in advance or may be lent to the wearer by a facility where the health checkup of the wearer is implemented. The smart watch 12 comprises sensors such as an image sensor, a temperature sensor, a pressure sensor, a vibration sensor, and a magnetic sensor, and has a biological information acquisition function of acquiring biological information (body temperature, pulse rate, blood pressure, blood glucose level, or the like) of the wearer by using information from these sensors. In addition, the smart watch 12 has a position information acquisition function of receiving radio waves from a global positioning system (GPS) satellite 18 and detecting a current position.

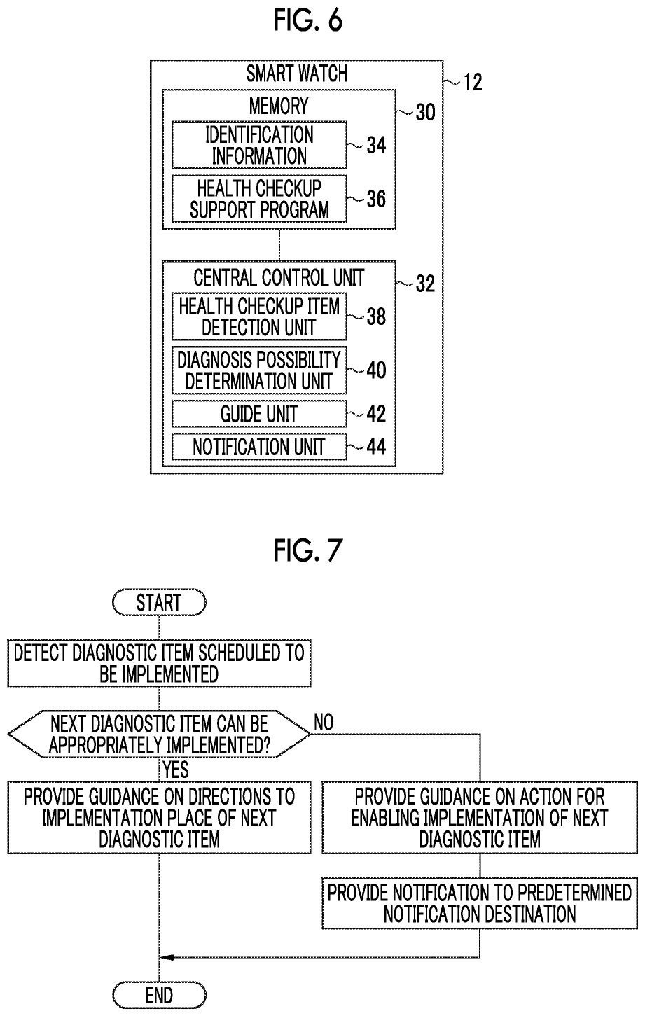

The biological information detected by a biological information detection function and the position information acquired by the position information acquisition function are transmitted to and stored in the history information server 13. The history information server 13 can be accessed by using the smart watch 12 or identification information 34 (see FIG. 6) for identifying the wearer, and history of the biological information or the current position of the wearer can be recognized by the accessing.

In the present embodiment, an example is described in which the history information server 13 stores the history of the biological information and the current position, but the history information server 13 may store history other than the biological information or the current position, such as the past injuries and diseases of the wearer of the smart watch 12. In addition, in the present embodiment, an example of applying the present invention to a smart watch as a wearable device is described, but the present invention may be applied to a wearable device other than the smart watch.

The health checkup information server 14 stores diagnostic item data 21, determination criterion data 22, health checkup progress information 23, and implementation place data 24 as health checkup information related to the health checkup.

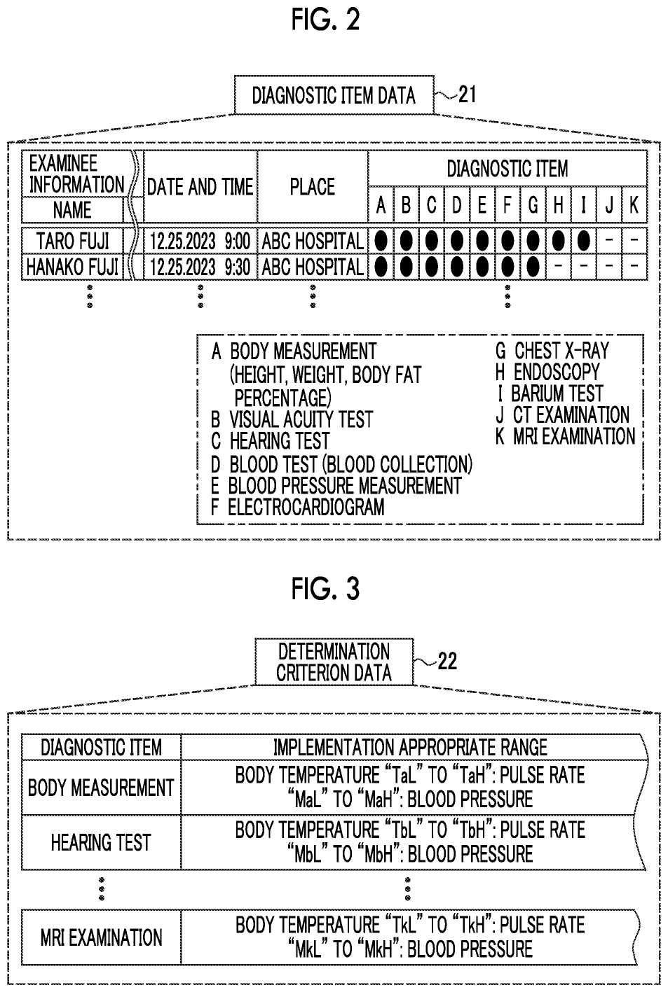

As shown in FIG. 2, the diagnostic item data 21 relates to a diagnostic item to be implemented in the health checkup, and the diagnostic item data 21 includes examinee information, a date and time, a place, and a diagnostic item. The examinee information is information about an examinee of the health checkup, and includes information indicating a name, an age, a sex, a height, a weight, and special notes (previous illness or chronic illness, injuries and diseases currently being treated, medicines prescribed and administered, and the like) of the examinee. The date and time is information indicating a date and time when the health checkup is scheduled to be implemented. The place is information indicating a place (such as a place of a medical facility) where the health checkup is scheduled to be implemented. The diagnostic item is information indicating a type of diagnosis to be implemented in the health checkup.

FIG. 2 shows an example in which information of two persons, a name “Taro Fuji” and a name “Hanako Fuji”, is stored as the diagnostic item data 21. Then, the name “Taro Fuji” has the contents of an examination date and time “9:00 AM, Dec. 25, 2023”, an examination place “ABC hospital”, and implementation (examination) of a health checkup for diagnostic items of “A to I” among diagnostic items of A to K. In addition, the name “Hanako Fuji” has the contents of an examination date and time “9:30 AM, Dec. 25, 2023”, an examination place “ABC hospital”, and implementation (examination) of a health checkup for diagnostic items of “A to G” among diagnostic items of A to K.

Here, the diagnostic item “A” is “body measurement”, the diagnostic item “B” is “visual acuity test”, the diagnostic item “C” is “hearing test”, the diagnostic item “D” is “blood test”, the diagnostic item “E” is “blood pressure measurement”, the diagnostic item “F” is “electrocardiogram”, the diagnostic item “G” is “chest X-ray”, the diagnostic item “H” is “endoscopy”, the diagnostic item “I” is “barium test”, the diagnostic item “J” is “CT examination”, and the diagnostic item “K” is “MRI examination”.

As shown in FIG. 3, in the determination criterion data 22, a range (appropriate range) of the biological information that can be considered to enable appropriate implementation is specified for each of the diagnostic items, and whether or not appropriate implementation is possible is determined based on whether or not the biological information is within the appropriate range. For example, in the example of FIG. 3, regarding the diagnostic item “body measurement”, an appropriate range of the biological information “body temperature” is specified as “TaL” or more and “TaH” or less. Therefore, in a case in which the “body temperature” is out of the appropriate range, it is determined that the “body measurement” cannot be appropriately implemented.

The appropriate range may be common regardless of an age, a sex, a previous illness, or the like, or may differ depending on an age, a sex, a previous illness, or the like. In addition, the appropriate range is not limited to the specification by a specific numerical value such as “body temperature” being “35 degrees” or more and “37 degrees” or less, and may be specified in consideration of the normal biological information of the examinee (the wearer of the smart watch 12) of the health checkup, such as “body temperature” being “minus 5% of normal temperature” or more and “plus 5% of normal temperature” or less. In this case, it is possible to extract the biological information of the examinee (the wearer of the smart watch 12) of the health checkup for a certain period in the past from the history information server 13 and use an average of values of the extracted biological information as the normal biological information.

As shown in FIG. 4, the health checkup progress information 23 is information indicating a progress status of the health checkup, in which a diagnostic item that has been implemented and a future examination schedule are associated with each other for each examinee of the health checkup. The examination schedule is a list of diagnostic items scheduled to be implemented in the future in the order of implementation (scheduled implementation order). In the present embodiment, an example will be described in which each diagnostic item is implemented in an alphabetical order indicating the items, but the implementation order of the diagnostic items can be changed as appropriate.

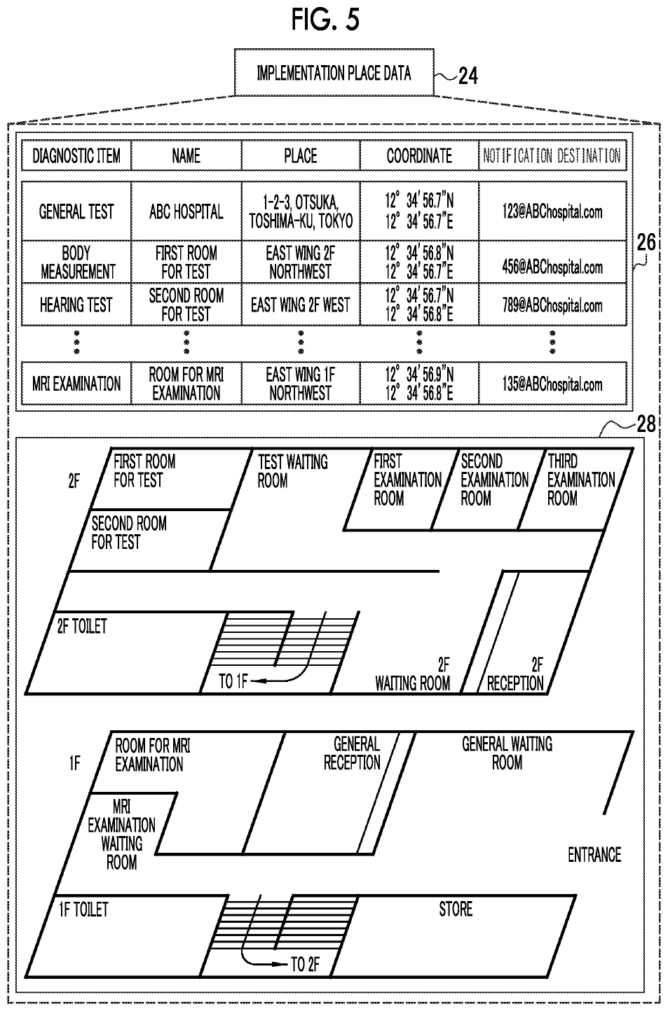

As shown in FIG. 5, the implementation place data 24 is information about an implementation place of the health checkup, and indicates a facility where the health checkup is implemented, an implementation place of each diagnostic item in the facility, and notification destination. In the present embodiment, the implementation place data 24 includes character information 26 indicating a facility where the health checkup is implemented and an implementation place of each diagnostic item, and map information 28 indicating a floor map of the facility where the health checkup is executed.

As shown in FIG. 6, the smart watch 12 is provided with a memory 30 and a central control unit 32. The identification information 34 is stored in the memory 30. The identification information 34 includes information for identifying the smart watch 12 and information for identifying the wearer of the smart watch 12.

In addition, the health checkup support program 36 is stored in the memory 30. The central control unit 32 functions as a health checkup item detection unit 38, a diagnosis possibility determination unit 40, a guide unit 42, and a notification unit 44 by operating the health checkup support program 36 stored in the memory 30.

The health checkup item detection unit 38 causes a computer (central control unit 32) of the smart watch 12 to realize a health checkup item detection function. The health checkup item detection unit 38 accesses the health checkup information server 14, refers to the diagnostic item data 21, and searches for an examinee (person scheduled to be examined) that matches the identification information 34. Then, the diagnostic item (the diagnostic item scheduled to be examined (implemented) on the day of the health checkup) of the searched examinee, that is, the wearer of the smart watch 12 is detected.

The diagnosis possibility determination unit 40 causes the computer (central control unit 32) of the smart watch 12 to realize a diagnosis possibility determination function. The diagnosis possibility determination unit 40 accesses the health checkup information server 14 to refer to the health checkup progress information 23, and, when the implementation time for each diagnostic item begins (or a predetermined time (for example, 10 minutes) before the implementation time begins), it is determined whether or not this diagnostic item (which is a diagnostic item scheduled to be implemented next, and is hereinafter referred to as a next diagnostic item) can be appropriately implemented. Specifically, the biological information of the wearer obtained by the smart watch 12 is compared with the determination criterion data 22, and it is determined whether or not the biological information is within an appropriate range of the next diagnostic item.

The guide unit 42 causes the computer (central control unit 32) of the smart watch 12 to realize a guidance function. The guide unit 42 provides guidance regarding implementation of the next diagnostic item based on a determination result of the diagnosis possibility determination unit 40.

The guidance regarding the implementation of the next diagnostic item includes guidance on directions to an implementation place of the next diagnostic item. The guidance on the directions is provided in a case in which the diagnosis possibility determination unit 40 determines that the biological information of the wearer of the smart watch 12 is within the appropriate range of the next diagnostic item. The guide unit 42 uses a current position of the wearer obtained by the smart watch 12 and information on the implementation place of the next diagnostic item obtained by the implementation place data 24 to provide guidance on directions from the current position to the implementation place of the diagnostic item, for example, by displaying the directions on a floor map of a facility where a health checkup is implemented.

In addition, the guidance regarding the implementation of the next diagnostic item includes guidance on an action for enabling implementation of the next diagnostic item. The guidance on the action is provided in a case in which the diagnosis possibility determination unit 40 determines that the biological information of the wearer of the smart watch 12 is out of the appropriate range of the next diagnostic item. The guide unit 42 provides guidance on an action for returning the biological information determined as being out of the appropriate range, to the appropriate range. Specifically, in a case in which a value of the heart rate is higher than the appropriate range, a message such as “please sit in a chair and relax” is displayed.

The notification unit 44 causes the computer (central control unit 32) of the smart watch 12 to realize a notification function. In a case in which the diagnosis possibility determination unit 40 determines that the biological information of the wearer of the smart watch 12 is out of the appropriate range of the next diagnostic item, the notification unit 44 notifies a predetermined notification destination of the fact. The predetermined notification destination is, for example, a terminal of a medical staff who is in charge of diagnosis of each diagnostic item, and is notification destination associated with the diagnostic item in the implementation place data 24 in the present embodiment. The notification unit 44 refers to the implementation place data 24 and notifies the notification destination corresponding to the diagnostic item determined as being out of the appropriate range of the fact.

Hereinafter, a processing flow of the health checkup support program 36 will be described with reference to FIG. 7. As shown in FIG. 7, in a case in which the health checkup support program 36 is executed, the diagnostic items of the health checkup scheduled to be implemented by the wearer of the smart watch 12 are detected, and, when the implementation time for each diagnostic item begins (or a predetermined time (for example, 10 minutes) before the implementation time begins), it is determined whether or not the next diagnostic item (which is a diagnostic item scheduled to be implemented next) can be appropriately implemented based on the biological information of the wearer. Then, in a case in which it is determined that the next diagnostic item can be appropriately implemented, guidance on the directions to the implementation place of the next diagnostic item is provided. On the other hand, in a case in which it is determined that the next diagnostic item cannot be appropriately implemented, guidance on an action for enabling the implementation of the next diagnostic item is provided. In addition, the predetermined notification destination is notified of the fact that the next diagnostic item cannot be appropriately implemented.

### Second Embodiment

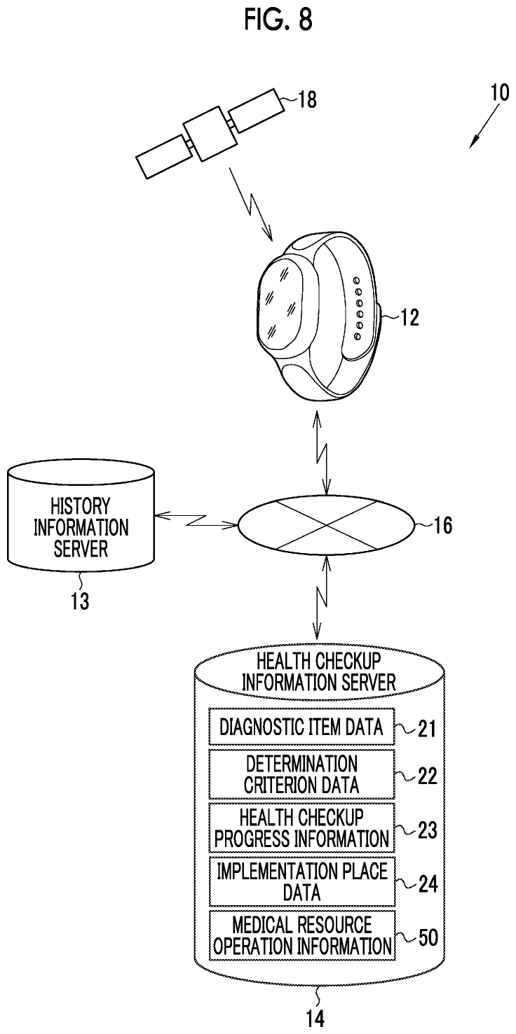

In a second embodiment, it is possible to decide and change the implementation order of the diagnostic items based on the biological information. As shown in FIG. 8, in the second embodiment, medical resource operation information 50 is stored in the health checkup information server 14. In the following description, the same members as those described above are denoted by the same reference numerals, and the description thereof will be omitted.

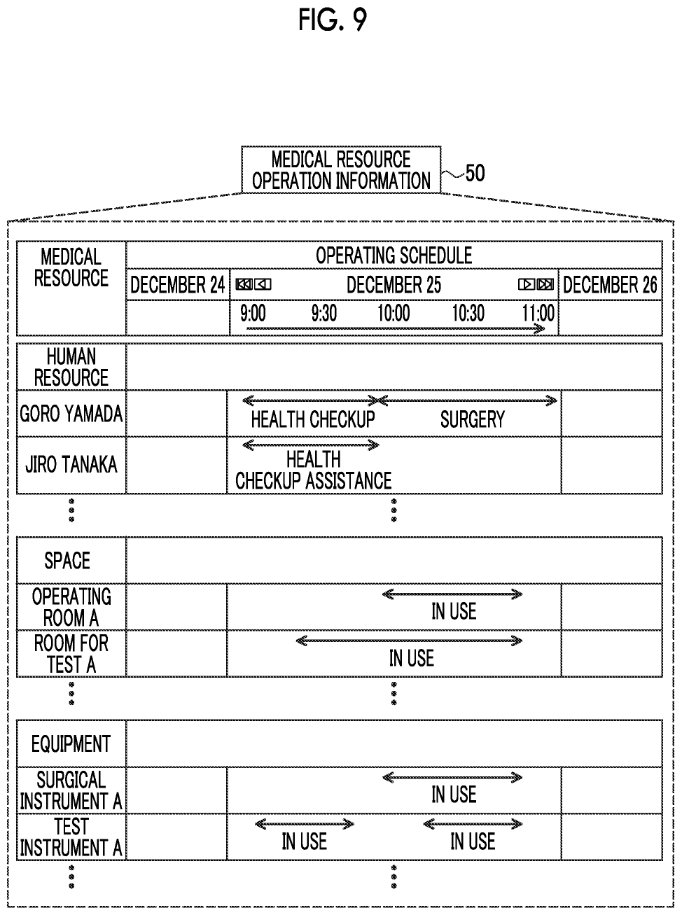

As shown in FIG. 9, the medical resource operation information 50 is information indicating an operating status (operating schedule) of medical resources in a facility where a health checkup is implemented. In the present embodiment, the facility where the health checkup is implemented is “ABC hospital”. In addition, the medical resources include, for example, a human resource such as a doctor and a nurse, a space such as an examination room and a room for test, and equipment such as a test instrument and a treatment tool.

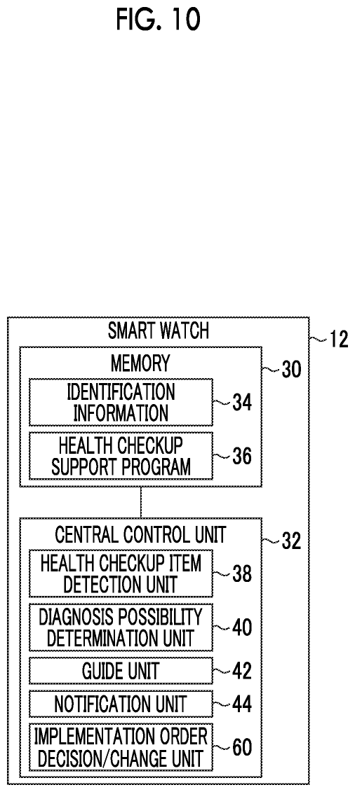

In addition, as shown in FIG. 10, in the second embodiment, by operating the health checkup support program 36 stored in the memory 30, the central control unit 32 functions as an implementation order decision/change unit 60 in addition to the above-described units. The implementation order decision/change unit 60 causes the computer (central control unit 32) of the smart watch 12 to realize an implementation order decision function and an implementation order change function.

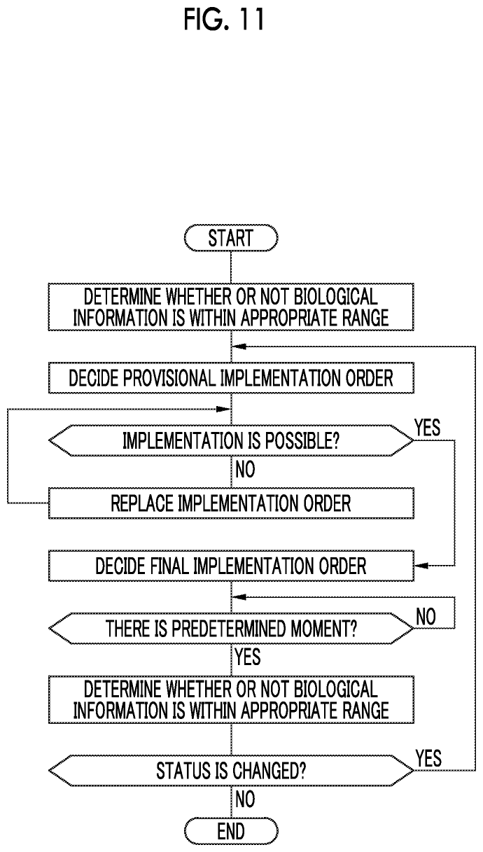

As shown in FIG. 11, in a case in which a scheduled implementation date and time of a health checkup, that is, an implementation time of a first diagnostic item comes, the implementation order decision/change unit 60 determines whether or not the biological information is within the appropriate range (whether or not appropriate implementation is possible) for all the diagnostic items, which are scheduled to be implemented, of the wearer of the smart watch 12. Then, an order obtained by arranging the diagnostic items that are within the appropriate range in a predetermined order (for example, an alphabetical order), and after these, by arranging the diagnostic items that are out of the appropriate range in a predetermined order (for example, an alphabetical order) is decided as a provisional implementation order.

Next, the implementation order decision/change unit 60 refers to the medical resource operation information 50, determines whether or not the implementation is possible in the provisional implementation order, and, in a case in which it is determined that the implementation is possible, this implementation order (provisional implementation order) is decided as a final implementation order. On the other hand, in the provisional implementation order, in a case in which it is determined that the implementation is impossible, the implementation order is replaced with an order in which the implementation is possible, and the replaced implementation order is decided as a final implementation order. In the replacement of the implementation order, it is permitted to perform the replacement only between the diagnostic items within the appropriate range and between the diagnostic items out of the appropriate range, and it is prohibited to perform the replacement between the diagnostic item within the appropriate range and the diagnostic item out of the appropriate range. In such a case, in the final implementation order as well, the implementation order of the diagnostic items within the appropriate range precedes the implementation order of the diagnostic items out of the appropriate range, as in the provisional implementation order. After that, the implementation order decision/change unit 60 updates the health checkup progress information 23 and the medical resource operation information 50 based on the final implementation order decided as described above.

In addition, even after the final implementation order is decided, the implementation order decision/change unit 60 determines whether or not the biological information is within the appropriate range (whether or not the appropriate implementation is possible) for the diagnostic item to be implemented in the future at a predetermined moment (for example, every predetermined time and/or each time one diagnostic item is implemented). Then, in a case in which the status is changed (in a case in which the diagnostic item that was within the appropriate range is out of the appropriate range or vice versa), the implementation order is changed. Specifically, a provisional implementation order is decided, and then a final implementation order is decided by the same method as the above-described method, and this (final implementation order) is set as an implementation order after change. Then, the health checkup progress information 23 and the medical resource operation information 50 are updated based on the final implementation order decided as described above.

In the above embodiment, a hardware structure of a processing unit that executes various kinds of processing, such as the health checkup item detection unit 38, the diagnosis possibility determination unit 40, the guide unit 42, the notification unit 44, and the implementation order decision/change unit 60, is various processors as shown below. The various processors include a central processing unit (CPU) that is a general-purpose processor that executes software (programs) to function as various processing units, a programmable logic device (PLD) that is a processor capable of changing a circuit configuration after manufacture, such as a field programmable gate array (FPGA), and an exclusive electric circuit that is a processor having a circuit configuration exclusively designed to execute various kinds of processing.

One processing unit may be configured of one of these various processors, or may be configured of a combination of two or more processors of the same type or different types (for example, a combination of a plurality of FPGAs or a combination of a CPU and an FPGA). In addition, a plurality of processing units may be configured of one processor. As an example in which the plurality of processing units are configured of one processor, first, as typified by computers such as a client or a server, one processor is configured of a combination of one or more CPUs and software, and this processor functions as the plurality of processing units. Second, as typified by a system on chip (SoC) or the like, a processor that realizes the functions of the entire system including the plurality of processing units by using one integrated circuit (IC) chip is used. As described above, the various processing units are configured using one or more of the various processors as a hardware structure.

Further, the hardware structure of these various processors is more specifically an electric circuit (circuitry) in a form in which circuit elements such as semiconductor elements are combined.

### EXPLANATION OF REFERENCES

10: health checkup support system
12: smart watch
13: history information server
14: health checkup information server (health checkup information storage unit)
16: network
18: GPS satellite
21: diagnostic item data
22: determination criterion data
23: health checkup progress information
24: implementation place data
26: character information
28: map information
30: memory
32: central control unit
34: identification information
36: health checkup support program
38: health checkup item detection unit
40: diagnosis possibility determination unit
42: guide unit
44: notification unit
50: medical resource operation information
60: implementation order decision/change unit

What is claimed is:

1. A non-transitory computer readable medium for storing a computer-executable program for causing a computer to function as a wearable device that acquires biological information of a wearer, the computer-executable program causing the computer to execute:
a diagnostic item detection function of detecting a diagnostic item on the day of a health checkup of the wearer by accessing a health checkup information storage that stores health checkup information related to the health checkup; and
a diagnosis possibility determination function of determining whether or not at least one of the diagnostic items detected by the diagnostic item detection function is appropriately implementable by using the biological information of the wearer obtained via the wearable device.

2. The non-transitory computer readable medium according to claim 1,
wherein the computer-executable program causes the computer to execute a guidance function of providing guidance regarding the implementation of the diagnostic item based on a determination result of the diagnosis possibility determination function.

3. The non-transitory computer readable medium according to claim 2,
wherein the guidance includes guidance on directions to an implementation place for a diagnostic item determined as being appropriately implementable.

4. The non-transitory computer readable medium according to claim 3,
wherein the health checkup information storage stores location information of the implementation place,
the wearable device acquires position information of the wearer, and
the guidance function is a function of providing the guidance using the location information of the implementation place and the position information of the wearer.

5. The non-transitory computer readable medium according to claim 2,
wherein the guidance includes guidance on an action for enabling implementation of a diagnostic item determined as not being appropriately implementable.

6. The non-transitory computer readable medium according to claim 1,
wherein the computer-executable program causes the computer to execute a notification function of, in a case in which it is determined that the diagnostic item is not appropriately implementable by the diagnosis possibility determination function, notifying a predetermined notification destination of the fact.

7. The non-transitory computer readable medium according to claim 6,
wherein the predetermined notification destination is a terminal of a staff of an implementation facility of the health checkup.

8. The non-transitory computer readable medium according to claim 1,
wherein, in a case in which a plurality of the diagnostic items are implemented in one health checkup,
the determination is performed for each of the plurality of diagnostic items by the diagnosis possibility determination function, and
the computer-executable program causes the computer to execute an implementation order decision function of deciding an implementation order of the plurality of diagnostic items by using a determination result of each of the plurality of diagnostic items.

9. The non-transitory computer readable medium according to claim 8,
wherein the determination by the diagnosis possibility determination function is repeatedly performed at a predetermined moment, and
the computer-executable program causes the computer to execute an implementation order change function of, in a case in which a diagnostic item determined as not being appropriately implementable in the determination occurs after the implementation order is decided, changing the implementation order.

10. The non-transitory computer readable medium according to claim 9,
wherein, in addition to the biological information of the wearer, a current operating status and a future operating schedule of a medical resource required for the implementation of each of the diagnostic items are used to decide the implementation order.

11. The program according to claim 1,
wherein the determination by the diagnosis possibility determination function is performed based on at least any of the past biological information of the wearer, an age, a sex, or a previous illness of the wearer.
